(19) United States
(12) Patent Application Publication
(10) Pub. No.: US 2008/0081851 A1
(43) Pub. Date: Apr. 3, 2008

(54) OPTICAL POLYMERS WITH HIGHER REFRACTIVE INDEX

(76) Inventors: Patrick H. Benz, Sarasota, FL (US); Jose A. Ors, Sarasota, FL (US)

Correspondence Address: FOLEY AND LARDNER LLP;SUITE 500, 3000 K STREET NW, WASHINGTON, DC, 20007, US

(21) Appl. No.: 11/849,234

(22) Filed: Aug. 31, 2007

Related U.S. Application Data
(60) Provisional application No. 60/841,549, filed on Sep. 1, 2006.

(51) Int. Cl. A61F 2/16; C07C 233/01; C08F 26/06

(52) U.S. Cl. 523/113; 526/296; 526/312; 564/161; 623/6.56

(57) ABSTRACT

Hydrophilic lens materials having a good combination of high refractive index and mechanical properties. The materials are clear and allow for use in increasingly narrow injectors in intraocular lens applications. A monomer based on a methacrylamide comprising a spacer and an aromatic group can be used to increase the refractive index. The mechanical properties are generally comparable with existing materials, and the optical properties are improved.

### RELATED APPLICATIONS

This application claims priority to U.S. provisional filing 60/841,549 filed Sep. 1, 2006, which is hereby incorporated by reference in its entirety.

### BACKGROUND

A need exists for better optical materials and in particular better lens materials including, for example, better polymer materials and better materials designed for use in or on an eye which provide properties for increasingly demanding applications, including intraocular lens materials (IOLs). See for example U.S. Pat. Nos. 7,067,602; 6,627,674; 6,566,417; 6,517,750; 6,267,784; 5,891,932; and 5,532,289 to Benz and Ors (Benz Research and Development Corporation). In many cases, a difficult balance of properties may be needed which require trading off one property for another. For example, optical properties and mechanical properties must be carefully balanced. Examples of important properties include equilibrium water content, refractive index, ability to fold and unfold, resilience, and clarity.

In some cases, high refractive index materials are desirable because this allows the material to be very thin in certain portions of a lens. A thin geometry facilitates folding and injection of lens materials through small incisions. However, attempts to prepare the proper optical materials can result in decreased mechanical performance, and vice versa. Moreover, undesired cloudiness can be found in some cases due to lack of compatibility or phase separation among the polymer components. In general, a need exists to find materials which can be adapted for use with known, useful lens materials to provide excellent additional properties without compromising existing good properties of the known materials.

In particular, U.S. Pat. No. 6,517,750 describes methods of making intraocular lens materials from a copolymer of a hydrophilic monomer and an alkoxyalkyl methacrylate. The lens is foldable so as to be insertable through a small incision in the eye. However, the patent does not teach or describe higher refractive index materials.

### SUMMARY

Presently, hydrated materials have been prepared which provide an excellent balance of optional properties, e.g, high refractive index, and good mechanical properties, e.g., folding behavior. Good clarity can be achieved. Combinations of properties can be achieved by control of the polymer structure.

In particular, one important embodiment is a hydrated polymer material comprising: (A) a crosslinked polymer comprising at least three monomeric subunits including: (i) at least one hydrophilic (meth)acrylate subunit, (ii) at least one alkoxyalkyl (meth)acrylate subunit, (iii) at least one (meth)acrylamide subunit comprising the (meth)acrylamide group, a spacer group covalently linked to the (meth)acrylamide group, and an aromatic group linked to the spacer group, and (iv) at least one crosslinker subunit; (B) water.

Also provided is a method of making a polymer material comprising: polymerizing a composition comprising at least three monomers in the presence of initiator to prepare a polymer comprising at least the following monomeric subunits: (i) at least one hydrophilic (meth)acrylate subunit, (ii) at least one alkoxyalkyl (meth)acrylate subunit, (iii) at least one (meth)acrylamide subunit comprising the (meth)acrylamide group, a spacer group covalently linked to the (meth)acrylamide group, and an aromatic group linked to the spacer group, and (iv) at least one crosslinker subunit.

Embodiments described herein include materials, polymers, whether hydrated materials and polymers or unhydrated materials and polymers, methods of making materials and polymers, methods of using materials and polymers, monomers useful for making polymers and materials, and shaped and engineered materials and polymers adapted for use in for example lenses and blanks for making of lenses.

### DETAILED DESCRIPTION

Introduction & Polymers

One skilled in the art can refer to U.S. Pat. Nos. 7,067,602; 6,627,674; 6,566,417; 6,517,750; 6,267,784; 5,891,932; and 5,532,289 for background in the practice of the presently described embodiments. In addition, materials and methods can be obtained from Benz Research and Development Corp., Sarasota, Fla. including various aspects of IOL technology materials. Additional technology is provided in U.S. provisional application Ser. No. 60/722,403 to Benz and Ors filed Oct. 3, 2005 “Polymers with High Refractive Index for Use in Intraocular Lens” which is hereby incorporated by reference in its entirety. The '403 provisional application describes preparation and machining of lens materials

Polymers, crosslinked polymers, copolymers, terpolymers, hydrogels, interpenetrating polymer networks, random versus block microstructures, oligomers, monomers, methods of polymerization and copolymerization, molecular weight, measurements, and related materials and technologies are generally known in the polymer arts and can be used in the practice of the presently described embodiments. See, for example, (1) Contemporary Polymer Chemistry, Allcock and Lamp, Prentice Hall, 1981, and (2) Textbook of Polymer Science, 3^{rd} Ed., Billmeyer, Wiley-Interscience, 1984. Free radical polymerization can be used to prepare the polymers herein.

Hydration of crosslinked polymers is known in the art in various technologies including hydrogel, membrane, and lens materials.

The term (meth)acrylate refers to both methacrylate or acrylate embodiments as understood by one skilled in the art. Methacrylate embodiments are preferred over acrylate embodiments.

The term (meth)acrylamide refers to both methacrylamide or acrylamide embodiments as understood by one skilled in the art. Methacrylamide embodiments are preferred over acrylamide embodiments.

EOEMA refers to ethoxyethyl methacrylate.

HEMA refers to 2-hydroxyethyl methacrylate.

TMPTMA refers to trimethylol propane trimethacrylate.

NBMA refers to N-benzyl methacrylamide.

PEMA refers to N-phenoxy ethyl methacrylamide.

BrPEM refers to brominated phenoxyethyl methacrylate.

IOLs made from hydrophilic materials such as hydrogels have shown appropriate biocompatibility in the eye. The water level can impart the right range of mechanical and optical properties that allow lenses to be easily folded, placed in small diameter injectors, and introduced into the capsular bag through incision sizes at or below 1.5 mm.

Polymer Material

The crosslinked polymer, as known in the art, can comprise various different monomeric subunits which can be represented by, for example:
-[A]ₐ−[B]_{b}—[C]_{c}−[D]_{d}- (I)
wherein for example A represents a hydrophilic monomeric subunit; B represents an alkoxyalkyl monomeric subunit, C represents a monomeric subunit comprising an aromatic group, and D represents a crosslinker monomeric subunit, and wherein the monomeric subunits are or are not randomly dispersed along the monomer chain. In some cases, some block character can be present in the distribution of monomeric subunits. The polymer chain can be a linear polymer chain apart from the crosslinker subunits which provide covalent or other types of linkage sites between the chains.

In a preferred embodiment of formula (I), the copolymer can be represented by:
—[CH₂CR¹(CH₃)]ₐ—[CH₂CR²(CH₃)]_{b}—[CH₂CR³(CH₃)]_{c}—[CH₂CR⁴(CH₃)]_{d}— (II)
wherein R¹ can be for example a group which imparts hydrophilic properties such as a polar group comprising oxygen or nitrogen atoms such as hydroxyl; or R² can be an alkoxyalkyl group; or R³ can be a group comprising a spacer and an aromatic group; or R⁴ can be a multifunctional group that binds to another polymer chain providing crosslinking points. If desired, one or more of the methyl groups bonded to the backbone can be replaced by a hydrogen (e.g., methacrylate versus acrylate; or methacrylamide versus acrylamide).

The end groups of the copolymer are not particularly limited but can be for example determined by the initiator used and the termination mechanisms present in the copolymerization reaction.

One embodiment is for a polymer which consists essentially of polymer subunits which substantially exclude those ingredients and subunits which compromise the basic and novel properties of the materials.

Polymerization conditions including initiator or catalyst selection can be selected to provide for clean polymerization so that for example there is little if any hydrogen abstraction or chain-transfer.

Aromatic Group Monomeric Subunits

The monomeric subunit comprising an aromatic group can be for example represented by M-S-A, wherein M represents the (meth)acrylamide group, S represents a spacer, and A represents an aromatic group. The subgroups M, S, and A can be covalently linked to each other. The purpose of the spacer is to link the acrylamide moiety with the aromatic group and provide a suitable combination of mechanical properties, including for example glass transition temperature, and solubility.

The aromatic group can be selected to increase the refractive index of the material. The aromatic groups can be hydrophobic.

The aromatic group can comprise one or more aromatic rings including for example one, two, or three or more aromatic rings. If desired, heterocyclic rings can be used such as naphthyl. Fused aromatic rings can be used. The number of carbon atoms can be for example C6 to C18, or C6 to C12.

In addition to hydrogen, aromatic rings can have substituents as desired including for example halogen atoms such as fluoro, chloro, bromo, or iodo. These substitutents are particularly useful if they can provide the electron density needed to increase the refractive index such as for example a bromo substitutent. Other substituents can comprise, for example, alkyl, alkoxy, alkyl hydroxyl groups. Substitution patterns can be ortho, meta, or para with respect to the binding of the aromatic moiety to the spacer group.

Bonding to the spacer group can be through a single bond.

The spacer group can be a linear chain of atoms connecting the (meth)acrylamide polymer backbone and the aromatic group and comprising for example one, two, three, four, five, or six atoms in the chain. Methylene units (—CH₂—) and oxygen atoms can be used. One skilled in the art can adjust the length of the spacer to adapt to the particular need and the other components of the formulation. If the spacer group becomes too large, in some embodiments the mechanical properties become undesirable. The atoms can be for example carbon atoms or can be a heteroatom such as oxygen. The linear chain can comprise substituents such as hydrogen. Examples of spacers include —CH₂— and —CH₂CH₂— and —CH₂CH₂OCH₂CH₂—.

In these embodiments, the methacrylamide portion can be hydrophilic while the spacer group and the aromatic group can be hydrophobic. The methacrylamide portion becomes part of a polypropylene backbone of the polymer with an amide group next to the backbone. This arrangement can prevent phase separation or cloudiness in the polymer by facilitating the presence of water near the polymer backbone.

The monomer can be further represented by the following structure: 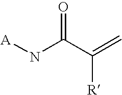
wherein R′ can be CH₃ or H, and A can be for example benzyl-, phenoxyethyl-, or phenoxyethoxyethyl-.

The aromatic or phenyl substitution can be represented by for example (circle inside ring omitted): 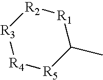
wherein R₁, R₂, R₃, R₄, or R₅ can be for example H, Br, Cl, Ph, Me, Et, and other alkyl, alkoxy, and alkyl hydroxy substituents.
Hydrophilic Monomeric Subunits

Monomeric subunits comprising hydrophilic moieties to provide hydrophilic properties are known in the art, see for example U.S. Pat. No. 6,517,750, which is hereby incorporated by reference in its entirety. For example, polar groups can be present comprising for example oxygen or nitrogen atoms, and groups capable of hydrogen bonding. This component facilitates water absorption. The amount of this component, along with amounts of more hydrophobic components, can be adjusted to provide a desired water uptake.

One embodiment makes use of HO—R-MA, wherein R is a spacer group between the HO-hydroxyl and the methacrylate, and R is an alkyl group of 1 to 6 carbon atoms.

A preferred material is 2-HEMA and in particular, highly pure forms of 2-HEMA, e.g, 99.9% pure, as the material may be in the eye for long periods of time. Content of ions and acid should be as close to zero as possible. For example, acidic impurities such as methacrylic acid can result in particle formation over time such as for example calcium phosphate particle formation.

Alkoxyalkyl Monomeric Subunits

Monomeric subunits which comprise alkoxyalkyl groups are known in the art, see for example U.S. Pat. No. 7,067,602 and U.S. Pat. No. 6,517,750, which are hereby incorporated by reference in its entirety.

One embodiment makes use of R2-O—R3-MA, wherein R2 and R3 can be independently an alkylene or alkyl group with 1 to 6 carbon atoms, and MA is methacrylate. The presence of this subunit provides advantageous mechanical properties to the polymer.

The amount of this subunit can be adjusted to control the amount of water uptake.

Crosslinker Subunits

Crosslinker subunits, including acrylates and methacrylates, are well-known in the art. They can be the result of crosslinking of difunctional or trifunctional or even tetrafunctional monomers such as a di(meth)acrylate or a tri(meth)acrylate. The crosslink density can also be controlled to control the amount of water present as well as the mechanical properties.

Monomers which can provide crosslinker subunits can be represented by R(X)ₙ, wherein R is a core organic group such as as a C2, or C3 or C4 or C5 or C6 group, with or without heteroatoms like oxygen, X is a reactive group such as acrylate or methacrylate, and n is the number of reactive groups such as 2, 3, or 4.

They can be prepared from a variety of multifunctional olefinic monomers such as, for example, ethyleneglycol dimethacrylate (EGDMA), trimethylol propane trimethacrylate (TMPTMA), trimethylol propane triacrylate (TMPTA), diethyleneglycoldimethacrylate (DiEGDMA), triethyleneglycoltrimethyacrylate (TriEGDMA), and the like. A preferred example is trimethylol propane trimethacrylate.

Amounts

The amounts of the various subunits can be adapted to provide the requisite balance of optical properties and mechanical properties, including hydrophobicity, clarity, folding ability, refractive index, and the like.

The amount of hydrophilic (meth)acrylate subunits can be for example about 50 wt. % to about 80 wt. %, or about 55 wt. % to about 75 wt. %, or about 60 wt. % to about 70 wt. %.

The amount of the alkoxyalkyl (meth)acrylate subunits can be for example about 10 wt. % to about 35 wt. %, or about 15 wt. % to about 30 wt. %.

The amount of the (meth)acrylamide subunits comprising the (meth)acrylamide group, the spacer group, and the aromatic group can be for example about 5 wt. % to about 25 wt. %, or about 10 wt. % to about 20 wt. %.

The amount of the crosslinker can be for example about 0.01 wt. % to about 2.5 wt. %, or about 0.1 wt. % to about 1.5 wt. %, or about 0.2 wt. % to about 1 wt. %.

The amounts of the initiator, before polymerization, is not particularly limited but can be for example about 0.02 wt. % to about 0.15 wt. %, or about 0.05 wt. % to about 0.10 wt. %.

The amounts of the subunits can in many cases be approximated by the amounts of the monomers used to make the polymer.

Water

The amount of water can be adapted to provide the desired optical and mechanical properties. The amount can be expressed in a weight percent with respect to the total amount of copolymer. The water content can be an equilibrium amount, or an amount that is substantially at equilibrium, e.g, within 10% of equilibrium.

The crosslinked polymer can absorb water and swell as known in the art. The water content can be for example about 5 wt. % to about 30 wt. %, or about 10 wt. to about 25 wt. %, or about 14 wt. % to about 22 wt. %.

Hydration with water also can remove residual impurities.

Refractive Index

Refractive index (RI) can be measured for the hydrated materials by methods known in the art which can include control of temperature. For example, RI can be measured at 20° C. or 35° C. wherein at the higher temperature the RI is lower. RI can be for example equal to or greater than 1.465, or equal to or greater than 1.47, or equal to or greater than 1.48. RI for dry materials can be measured but provide much higher values as water lowers RI. The refractive index upper limit is not particularly limited but can be for example 1.55 or 1.52. Hence, exemplary ranges include for example 1.465 to 1.55 or 1.47 to 1.52.

Refractive indices are described further in the working examples.

Folding

Folding tests and resultant optical properties are described further in the working examples. Lenses unfold in for example about 10 seconds to about 120 seconds, or about 10 seconds to about 90 seconds when placed in saline solution at 35-37° C. Upon folding the lens, the time needed to mechanically regain the original shape and also to regain optical properties can be measured. The average center thickness (CT) of the lens can be generally less than 1 mm.

In a preferred embodiment, the hydrated polymer provides an unfolding time of about 10 seconds to about 120 seconds in 35° C. saline.

Other Properties

An important aspect is to provide combinations of two or more properties such as for example a combination of sufficiently high refractive index as well as folding property. A third property can be optical clarity.

Other properties which can be tailored include residual HEMA, radial expansion, linear expansion, % transmission at 600 nm and at 400 nm, glass transition temperature (Tg), isotropic expansion with hydration, pore size, modulus of elasticity, tensile strength.

Blank size can be characterized by diameter and thickness. For example, diameter can be about 10 mm to about 20 mm, for about 12 mm to about 16 mm. Thickness can be for example about 2 mm to about 4 mm, or about 2.5 mm to about 3.5 mm.

Making Lenses

The preparation of lenses is known in the art. See for example U.S. Pat. No. 7,067,602, which is hereby incorporated by reference. FIGS. 1 and 2 therein describe different shaped lens including lens having a plate-shaped haptic and lens having a C-shaped haptic.

Method of Making the Aromatic Monomer

The aromatic monomer can be obtained and purified by methods known in the art including use of condensation and addition reactions, coupling polymerization monomer with side group moieties, for example. For example, a phenoxyethyl methacrylamide monomer can be obtained from the condensation reaction of 2-phenoxyethyl amine and methacroyl chloride or methacrylic anhydride, both commercially available from Aldrich Chemical Company, using methods common to the art. The material can be purified via known methods such as vacuum distillation to yield a colorless liquid. Purity can be determined using gas chromatography.

Color

U.S. Provisional application Ser. Nos. 60/893,065 filed Mar. 5, 2007 and 60/914,597 filed Apr. 27, 2007 to Benz and Ors are hereby incorporated by reference and can be used in the practice of the inventions including the claims and figures. These applications teach chromophores and linking groups for same.

For example, 60/893,065 describes use of compositions comprising a polymerizable vinyl group covalently linked to a benzene ring-based chromophore comprising a ketone at the 1-position, a substituted or unsubstituted amino group at the 2-position, and an oxygen atom at the 3-position of the benzene ring, wherein the chromophore does not comprise an oxidantive polymerization product of 3-hydroxykynurenine.

In addition, 60/914,597 describes use of, for example, compositions comprising a compound comprising a polymerizable vinyl group covalently linked to a benzene ring-based chromophore comprising: a substituent comprising a carbonyl group at the 1-position of the benzene ring; a substituent comprising a hydroxy, ether, ester or a combination thereof at the 2-position of the benzene ring; and a substituent comprising a p-nitrophenylazo group at the 4-position of the benzene ring, including salts thereof.

These chromophores can be used to tune the color to mimic natural eye color including aging impacts on eye color. The chromophore can be covalently bound to monomers and polymers as described herein, and introduced into a polymer structure by copolymerization.

### WORKING EXAMPLES

Non-limiting working examples are provided.

Part I

### Working Examples 1-8

Table 1 shows some example polymer formulas along with their refractive index and water content. Sample 7 is comparative. Exemplary synthetic procedures and lens formation are also provided for three samples.

TABLE I

| | 2- | | | | | | | N_{D} @ | N_{D} @ | |
|---|---|---|---|---|---|---|---|---|---|---|
| Sample | Hema | EOEMA | NBMA | PEMA | BrPEM | TMPTMA | Initiator | 20° C. | 35° C. | H20 % |
| 1 (162) | 65 | 17 | 0 | 18 | 0 | 0.5 | 0.07 | 1.4822 | 1.481 | 15.0 |
| 2 (158-1) | 65 | 20 | 0 | 15 | 0 | 0.5 | 0.07 | 1.4745 | 1.4740 | 15.7 |
| 3 (158-2) | 65 | 18 | 0 | 17 | 0 | 0.5 | 0.07 | 1.4803 | 1.4799 | 14.9 |
| 4 (158-3) | 65 | 16 | 0 | 19 | 0 | 0.5 | 0.07 | 1.4835 | 1.4825 | 16.2 |
| 5 (137-1) | 62.3 | 25.2 | 12.5 | 0 | 0 | 0.5 | 0.07 | 1.4806 | 1.4798 | 17.8 |
| 6 (137-2) | 64.8 | 22.7 | 12.5 | 0 | 0 | 0.5 | 0.07 | 1.4793 | 1.4784 | 18.6 |
| 7 (126-1) | 65 | 20 | 5 | 0 | 10 | 0.5 | 0.07 | 1.4755 | 1.4760 | 20.8 |
| 8 (126-2) | 64.8 | 25.2 | 10 | 0 | 0 | 0.5 | 0.07 | 1.4772 | 1.4765 | 20.5 |

### Example 1

### Preparation of HEMA/EOEMA/PEMA Copolymer with 15% Water Content

A mixture of 65 grams of 2-HEMA, 17 grams of EOEMA and 18 grams of PEMA were mixed with and 0.07 grams of 2,2-azobis (2,4-dimethylvaleronitrile) were added. The total diester concentration was adjusted to 0.5% by weight with TMPTMA. The mixture was degassed while applying vigorous stirring. The mixture was dispensed into cylindrical molds, polymerized at 24° C. for 28 hours, and post-cured at 118° C. for 5 hours. The polymer was then removed from the molds and formed into contact lens blanks as known in the art. The mechanical formation process comprises cutting the polymer into cylinders of 0.5 to 0.65 inches (1.27 to 1.65 cm.) in diameter and 0.1 to 0.2 inches (0.25 to 0.51 cm.) in thickness.

### Example 5

### Preparation of HEMA/EOEMA/NBMA Copolymer with 18% Water Content

A mixture comprising 62.3 grams of 2-HEMA, 25.2 grams of EOEMA and 12.5 grams of NBMA were mixed with and 0.07 grams of 2,2-azobis (2,4-dimethylvaleronitrile) were added. The total diester concentration was adjusted to 0.5% by weight with TMPTMA. The mixture was degassed while applying vigorous stirring. The mixture was dispensed into cylindrical molds, polymerized at 24° C. for 28 hours, and post-cured at 118° C. for 5 hours. The polymer was then removed from the molds and formed into contact lens blanks as known in the art. The mechanical formation process comprises cutting the polymer into cylinders of 0.5 to 0.65 inches (1.27 to 1.65 cm.) in diameter and 0.1 to 0.2 inches (0.25 to 0.51 cm.) in thickness.

### Example 7

### Preparation of HEMA/EOEMA/BrPEM Copolymer with 21% Water Content

A mixture comprising 65 grams of 2-HEMA, 20 grams of EOEMA and 5 grams of BrPEM were mixed with and 0.07 grams of 2,2-azobis (2,4-dimethylvaleronitrile) were added. The total diester concentration was adjusted to 0.5% by weight with TMPTMA. The mixture was degassed while applying vigorous stirring. The mixture was dispensed into cylindrical molds, polymerized at 24° C. for 28 hours, and post-cured at 118° C. for 5 hours. The polymer was then removed from the molds and formed into contact lens blanks as known in the art. The mechanical formation process comprises cutting the polymer into cylinders of 0.5 to 0.65 inches (1.27 to 1.65 cm.) in diameter and 0.1 to 0.2 inches (0.25 to 0.51 cm.) in thickness.

Part II—Test Procedure for Folding-Unfolding Evaluation

Samples: One-piece lenses made in standard C-loop design

General Procedure: Test lenses were stored in vials containing pH=7.2 buffered saline at 20° C. for 48 hours. Prior to folding, both the Diopter power and MTF for each test lens is recorded using an IOLA. After measurement, each lens is returned to its corresponding vial.

Using a coated tweezers, the lens was gently removed from the vial. Using another pair of tweezers, the lens was gently folded along the haptic axis and held for 2 to 3 seconds. The folded lens was then released unto a Petri-dish containing the buffered saline that had been heated to 35 to 37° C. Unfolding time was measured from the point of release to the time the lens has regained the original flat (180°) geometry. The time measurement was repeated using two additional lenses and the average reported.

Once each lens unfolded, it was placed in the IOLA to record the time for recovery of power and MTF.

Test results are reported in Table 1. Table 2 shows comparative numbers for current hydrophilic IOL products. The IOL25 product is the material model for the mechanical properties of the current invention.

TABLE II
Folding-Unfolding Test

| | | | TIME TO UNFOLD | TIME TO REGAIN | | |
|---|---|---|---|---|---|---|
| | | | IN 35° C. SALINE | OPTICS IN 35° C. | | |
| Sample # | POWER | MTF | (min) | (min) | POWER | MTF |
| 1 | 18.7 | 0.48 | 0.33 | | 19.1 | 0.63 |
| 2 | 19.1 | 0.50 | 0.33 | | 19.0 | 0.49 |
| 3 | 19.1 | 0.64 | 0.33 | 2.13 | 19.0 | 0.62 |
| 4 | 19.1 | 0.64 | 0.33 | 2.20 | 19.2 | 0.47 |
| 5 | 19.2 | 0.62 | 0.33 | 1.38 | 19.1 | 0.61 |
| 6 | 19.2 | 0.64 | 0.33 | 3.01 | 19.0 | 0.61 |
| 7 | 19.2 | 0.64 | 0.30 | 1.12 | 19.1 | 0.64 |
| 8 | 18.8 | 0.32 | 0.33 | 1.45 | 18.7 | 0.32 |
| AVG | 19.0 | 0.56 | 0.3 | 1.9 | 19.0 | 0.55 |
| SD | 0.18 | 0.12 | | | 0.14 | 0.11 |
| 1 | 24.5 | 0.64 | 0.42 | 2.36 | 24.3 | 0.53 |
| 2 | 24.7 | 0.55 | 0.45 | 2.13 | 24.5 | 0.48 |
| 3 | 24.7 | 0.55 | 0.40 | 1.32 | 24.6 | 0.40 |
| 4 | 24.6 | 0.44 | 0.43 | 3.51 | 24.6 | 0.42 |
| 5 | 24.6 | 0.49 | 0.40 | 1.42 | 24.6 | 0.44 |
| 6 | 24.6 | 0.45 | 0.38 | 1.48 | 24.6 | 0.41 |
| 7 | 24.7 | 0.52 | 0.48 | 1.42 | 24.6 | 0.40 |
| AVG | 24.6 | 0.52 | 0.4 | 1.9 | 24.5 | 0.44 |
| SD | 0.07 | 0.07 | | | 0.10 | 0.05 |

TABLE III
Folding-Unfolding Comparison with Current Hydrophilic Products

| | | | | OPTICS | |
|---|---|---|---|---|---|
| | | | TIME TO UNFOLD | IN 35° C. | |
| | | | IN 35° C. SALINE | AFTER 5 | |
| Sample # | POWER | MTF | (min) | min | MTF |
| IOL25 | 19.9 | 0.56 | 0.20 | 20.05 | 0.53 |
| BF26 | 19.15 | 0.62 | 0.45 | 19.17 | 0.56 |

Part III—Folding-Unfolding Comparison with Current Hydrophilic Product IOL25

TABLE IV

| | | | | | | |
|---|---|---|---|---|---|---|
| | | | TIME TO UNFOLD | TIME TO REGAIN | | |
| | | | IN 35° C. SALINE | OPTICS IN 35° C. | | |
| Sample # | POWER | MTF | (min) | (min) | POWER | MTF |

I25CL TARGET DIOPTER 18.00

| | | | | | | |
|---|---|---|---|---|---|---|
| 1 | 17.8 | 0.52 | 0.2 | 3.5 | 17.9 | 0.58 |
| 2 | 18.2 | 0.57 | 0.18 | 2.2 | 18.2 | 0.50 |
| 3 | 18.1 | 0.57 | 0.28 | 3.5 | 18.2 | 0.44 |
| AVG | 18.0 | 0.55 | 0.22 | 3.1 | 18.1 | 0.51 |
| STDV | 0.24 | 0.03 | 0.05 | 0.7 | 0.12 | 0.07 |

I25CLU TARGET DIOPTER 23.00

| | | | | | | |
|---|---|---|---|---|---|---|
| 1 | 23.2 | 0.55 | 0.22 | 0.7 | 23.1 | 0.55 |
| 2 | 23.2 | 0.64 | 0.18 | 0.5 | 23.1 | 0.53 |
| 3 | 23.2 | 0.56 | 0.15 | 1.0 | 23.00 | 0.45 |
| AVG | 23.2 | 0.58 | 0.18 | 0.73 | 23.1 | 0.51 |
| STDV | 0.01 | 0.05 | 0.04 | 0.25 | 0.04 | 0.05 |

U.S. priority provisional application 60/841,549 describes the following 55 numbered emobidments:

Embodiment 1. A hydrated polymer material comprising:

A. a crosslinked polymer comprising at least three monomeric subunits including:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit;

B. water.

2. The hydrated polymer material according to embodiment 1, wherein the hydrated polymer material has refractive index of at least 1.465 at 20° C.

3. The hydrated polymer material according to embodiment 1, wherein the hydrated polymer material has refractive index of at least 1.48 at 20° C.

4. The hydrated polymer material according to embodiment 1, wherein the hydrated polymer material is in a C-loop design and can be folded along the haptic axis without mechanical rupture.

5. The hydrated polymer material according to embodiment 1, wherein the hydrated polymer provides an unfolding time of about 10 seconds to about 120 seconds in 35° C. saline.

6. The hydrated polymer material according to embodiment 1, wherein the hydrophilic (meth)acrylate subunit comprises an oxygen atom in the side group.

7. The hydrated polymer material according to embodiment 1, wherein the hydrophilic (meth)acrylate subunit comprises hydroxyl side group.

8. The hydrated polymer material according to embodiment 1, wherein the alkoxyalkyl (meth)acrylate subunit comprises an ethoxy or propoxy group.

9. The hydrated polymer material according to embodiment 1, wherein the alkoxyalkyl (meth)acrylate subunit comprises a methylene, ethylene, or propylene alkyl.

10. The hydrated polymer material according to embodiment 1, wherein the aromatic group comprises a phenyl ring.

11. The hydrated polymer material according to embodiment 1, wherein the aromatic group comprises an alkyl, alkoxy or alkyl aromatic substituent.

12. The hydrated polymer material according to embodiment 1, wherein the aromatic group comprises a bromine aromatic substituent.

13. The hydrated polymer material according to embodiment 1, wherein the spacer group comprises an alkylene spacer group or an alkyleneoxy spacer group.

14. The hydrated polymer material according to embodiment 1, wherein the crosslinker subunit comprises two or more crosslinking sites in the side group.

15. The hydrated polymer material according to embodiment 1, wherein the (meth)acrylate and (meth)acrylamide groups in (i), (ii), and (iii) are methacrylate and methacrylamide respectively.

16. The hydrated polymer material according to embodiment 1, wherein the water content is at least about 10 wt. %.

17. The hydrated polymer material according to embodiment 1, wherein the water content is at least about 15 wt. %.

18. The hydrated polymer material according to embodiment 1, wherein the water content is at least 10 wt. % but less than about 25 wt. %.

19A. The hydrated polymer material according to embodiment 17, wherein the amount of hydrophilic (meth)acrylate subunit is about 50 wt. % to about 80 wt. %; the amount of the alkoxyalkyl (meth)acrylate subunit is about 10 wt. % to about 35 wt. %; the amount of the (meth)acrylamide subunit is about 5 wt. % to about 25 wt. %; and the amount of the crosslinker is about 0.01 wt. % to about 2.5 wt. %.

19B. The hydrated polymer material according to embodiment 14, wherein the amount of hydrophilic (meth)acrylate subunit is about 60 wt. % to about 70 wt. %; the amount of the alkoxyalkyl (meth)acrylate subunit is about 15 wt. % to about 30 wt. %; the amount of the (meth)acrylamide subunit is about 10 wt. % to about 20 wt. %; and the amount of the crosslinker is about 0.2 wt. % to about 1 wt. %.

20. The hydrated polymer according to embodiment 19, wherein the refractive index is at least 1.465, wherein the hydrophilic (meth)acrylate subunit comprises hydroxyl side group, wherein the alkoxyalkyl(meth)acrylate subunit comprises an ethoxy group, wherein the spacer group comprises an alkylene spacer group or an alkyleneoxy spacer group, and the crosslinker subunit comprises two or more crosslinking sites in the side group.

Embodiment 21. A method of making a polymer material comprising:

polymerizing a composition comprising at least three different monomers in the presence of initiator to prepare a polymer comprising at least the following monomeric subunits:

(i) at least one hydrophilic (meth)acrylate subunit,

(ii) at least one alkoxyalkyl (meth)acrylate subunit,

(iii) at least one (meth)acrylamide subunit comprising the (meth)acrylamide group, a spacer group covalently linked to the (meth)acrylamide group, and an aromatic group linked to the spacer group, and

(iv) at least one crosslinker subunit.

22. The method according to embodiment 21, further comprising the step of hydrating the polymer.

23. The method according to embodiment 21, further comprising the step of hydrating the polymer to provide a refractive index of 1.465 at 20° C.

24. The method according to embodiment 21, further comprising the step of hydrating the polymer to provide a refractive index of 1.48 at 20° C.

25. The method according to embodiment 21, further comprising the step of hydrating the polymer to a water content of at least about 10 wt. %.

26. The method according to embodiment 21, further comprising the step of hydrating the polymer to a water content of at least about 15 wt. %.

27. The method according to embodiment 21, further comprising the step of hydrating the polymer to a water content of at least about 10 wt. % but less than about 25 wt. %.

28. The method according to 21, wherein the hydrophilic (meth)acrylate subunit comprises an oxygen atom in the side group.

29. The method according to embodiment 21, wherein the hydrophilic (meth)acrylate subunit comprises hydroxyl side group.

30. The method according to embodiment 21, wherein the alkoxyalkyl (meth)acrylate subunit comprises an ethoxy group.

31. The method according to embodiment 21, wherein the alkoxyalkyl (meth)acrylate subunit comprises an ethylene group as alkyl.

32. The method according to embodiment 21, wherein the aromatic group comprises a phenyl ring.

33. The method according to 21, wherein the aromatic group comprises a bromine aromatic substituent.

34. The hydrated polymer material according to embodiment 1, wherein the aromatic group comprises an alkyl, alkoxy or alkyl hydroxyl aromatic substituent.

35. The method according to embodiment 21, wherein the spacer group comprises an alkylene spacer group or an alkyleneoxy spacer group.

36. The method according to embodiment 21, wherein the crosslinker subunit comprises two or more crosslinking sites in the side group.

37. The method according to embodiment 21, wherein the (meth)acrylate and (meth)acrylamide groups in (i), (ii), and (iii) are methacrylate and methacrylamide respectively.

38. The method according to embodiment 21, wherein the amount of hydrophilic (meth)acrylate subunit is about 50 wt. % to about 80 wt. %; the amount of the alkoxyalkyl (meth)acrylate subunit is about 10 wt. % to about 35 wt. %; the amount of the (meth)acrylamide subunit is about 5 wt. % to about 25 wt. %; and the amount of the crosslinker is about 0.01 wt. % to about 2.5 wt. %.

39. The method according to embodiment 21, further comprising the step of hydrating the polymer, wherein the refractive index of the hydrated polymer is at least 1.465, wherein the hydrophilic (meth)acrylate subunit comprises hydroxyl side group, wherein the alkoxyalkyl(meth)acrylate subunit comprises an ethoxy group, wherein the spacer group comprises an alkylene spacer group or an alkyleneoxy spacer group, and the crosslinker subunit comprises two or more crosslinking sites in the side group.

40. The method according to embodiment 39, wherein the aromatic group is phenyl.

Embodiment 41. A monomer compound comprising a (meth)acrylamide group, a spacer group covalently linked to the (meth)acrylamide group, and an aromatic group linked to the spacer group.

42. The compound according to embodiment 41, wherein the aromatic group comprises a phenyl ring.

43. The compound according to embodiment 41, wherein the aromatic group comprises a halogenated aromatic substituent.

44. The compound according to embodiment 41, wherein the aromatic group comprises a bromine aromatic substituent.

45. The compound according to embodiment 41, wherein the aromatic group is phenyl.

46. The compound according to embodiment 41, wherein the spacer group comprises an alkylene spacer group or an alkyleneoxy spacer group.

47. The compound according to embodiment 41, wherein the spacer group comprises an oxygen atom.

48. The compound according to embodiment 41, wherein the spacer group comprises a —CH₂—group or linear chain of carbon atoms.

49. The compound according to embodiment 41, wherein the aromatic group is a C6 moiety and the spacer is a C2 moiety.

50. The compound according to embodiment 41, wherein the (meth)acrylamide group is a methacrylamide group.

Embodiment 51. A polymer comprising at least one (meth)acrylamide subunit, wherein the subunit comprises a (meth)acrylamide group, a spacer group covalently linked to the (meth)acrylamide group, and an aromatic group linked to the spacer group.

52. The polymer according to embodiment 51, wherein the polymer is hydrated with water to provide a refractive index of at least 1.465 at 20 C.

Embodiment 53. An intraocular lens comprising a hydrated polymer material comprising:

A. a crosslinked polymer comprising at least four monomeric subunits including:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit;

B. water.

Embodiment 54. A hydrated polymer material consisting essentially of:

A. a crosslinked polymer consisting essentially of at least three monomeric subunits including:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit;

B. water.

Embodiment 55. A method of making a polymer material consisting essentially of:

polymerizing a composition consisting essentially of at least three different monomers in the presence of initiator to prepare a polymer comprising at least the following monomeric subunits:

(i) at least one hydrophilic (meth)acrylate subunit,

(ii) at least one alkoxyalkyl (meth)acrylate subunit,

(iii) at least one (meth)acrylamide subunit comprising the (meth)acrylamide group, a spacer group covalently linked to the (meth)acrylamide group, and an aromatic group linked to the spacer group, and

(iv) at least one crosslinker subunit.

What is claimed is:

1. A hydrated polymer material comprising:
A. a crosslinked polymer comprising at least three monomeric subunits including:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit;
B. water.

2. The hydrated polymer material according to claim 1, wherein the hydrated polymer material has refractive index of at least 1.465 at 20° C.

3. The hydrated polymer material according to claim 1, wherein the hydrated polymer material has refractive index of at least 1.48 at 20° C.

4. The hydrated polymer material according to claim 1, wherein the hydrophilic (meth)acrylate subunit comprises an oxygen atom in the side group.

5. The hydrated polymer material according to claim 1, wherein the hydrophilic (meth)acrylate subunit comprises hydroxyl side group.

6. The hydrated polymer material according to claim 1, wherein the alkoxyalkyl (meth)acrylate subunit comprises an ethoxy or propoxy group.

7. The hydrated polymer material according to claim 1, wherein the aromatic group comprises a phenyl ring.

8. The hydrated polymer material according to claim 1, wherein the aromatic group comprises an alkyl, alkoxy or alkyl aromatic substituent.

9. The hydrated polymer material according to claim 1, wherein the water content is at least about 10 wt. %.

10. The hydrated polymer material according to claim 1, wherein the polymer further comprises a covalently bound chromophore.

11. A method of making a polymer material comprising:
polymerizing a composition comprising at least three different monomers in the presence of initiator to prepare a polymer comprising at least the following monomeric subunits:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit.

12. The method according to claim 11, further comprising the step of hydrating the polymer.

13. The method according to claim 11, further comprising the step of hydrating the polymer to provide a refractive index of 1.465 at 20° C.

14. The method according to claim 11, further comprising the step of hydrating the polymer to provide a refractive index of 1.48 at 20° C.

15. The method according to claim 11, further comprising the step of hydrating the polymer to a water content of at least about 10 wt. %.

16. The method according to claim 11, further comprising the step of hydrating the polymer to a water content of at least about 15 wt. %.

17. The method according to claim 11, further comprising the step of hydrating the polymer to a water content of at least about 10 wt. % but less than about 25 wt. %.

18. The method according to claim 11, wherein the hydrophilic (meth)acrylate subunit comprises an oxygen atom in the side group.

19. The method according to claim 11, wherein the hydrophilic (meth)acrylate subunit comprises hydroxyl side group.

20. The method according to claim 11, wherein the alkoxyalkyl (meth)acrylate subunit comprises an ethoxy group.

21. The method according to claim 11, wherein the aromatic group comprises a phenyl ring.

22. The method according to claim 11, wherein the aromatic group comprises a bromine aromatic substituent.

23. A monomer compound comprising a (meth)acrylamide group, a spacer group covalently linked to the (meth)acrylamide group, and an aromatic group linked to the spacer group.

24. A polymer comprising at least one (meth)acrylamide subunit, wherein the subunit comprises a (meth)acrylamide group, a spacer group covalently linked to the (meth)acrylamide group, and an aromatic group linked to the spacer group.

25. An intraocular lens comprising a hydrated polymer material comprising:
A. a crosslinked polymer comprising at least four monomeric subunits including:
(i) at least one hydrophilic (meth)acrylate subunit,
(ii) at least one alkoxyalkyl (meth)acrylate subunit,
(iii) at least one (meth)acrylamide subunit comprising the (meth)acrylamide group, a spacer group covalently linked to the (meth)acrylamide group, and an aromatic group linked to the spacer group, and
(iv) at least one crosslinker subunit;
B. water.
